(19) United States
(12) Patent Application Publication
(10) Pub. No.: US 2022/0040069 A1
(43) Pub. Date: Feb. 10, 2022

(54) TITANIUM DIOXIDE DISPERSION

(71) Applicant: Croda International Plc, Goole East Yorkshire (GB)

(72) Inventors: Amy Rose Goddard, Goole East Yorkshire (GB); Bhaven Chavan, Goole East Yorkshire (GB); Robert Michael Sayer, Goole East Yorkshire (GB); Ian Robert Tooley, Goole East Yorkshire (GB)

(73) Assignee: Croda International Plc, Goole East Yorkshire (GB)

(21) Appl. No.: 17/279,886

(22) PCT Filed: Oct. 4, 2019
(86) PCT No.: PCT/EP2019/076942

§ 371 Date: Mar. 25, 2021

(30) Foreign Application Priority Data
Oct. 12, 2018 (GB) 1816643.9

(51) Int. Cl. A61K 8/29; C01G 23/08; C09C 1/36; B01F 17/00; A61Q 17/04; A61K 8/85

(52) U.S. Cl. CPC A61K 8/29; C01G 23/08; C09C 1/3623; B01F 17/0028; C01P 2002/82; A61K 8/85; C01P 2004/62; C01P 2004/61; A61Q 17/04

(57) ABSTRACT

The invention provides a dispersion of precursor titanium dioxide particles having an intensity mean peak value particle size, measured by DLS, or a number mean peak value particle size, measured by DLS, in the range from 0.2 to 2.0 μm within a dispersing medium, wherein the particle size distribution of the titanium dioxide particles in dispersion is narrower than the particle size distribution of the precursor titanium dioxide particles. There is also provided the use of the dispersion of titanium dioxide particles to attenuate infrared radiation, particularly in a cosmetic composition, more particularly a cosmetic composition to provide protection against IR radiation, more particularly IRA radiation. There is also provided a personal care composition comprising the titanium dioxide dispersion.

### FIELD OF INVENTION

The present invention relates to dispersions of titanium dioxide particles and a method of making thereof. The invention further relates to the use of the said dispersions in personal care products, in particular for the attenuation of infrared radiation.

### BACKGROUND

Solar radiation that reaches earth's surface contains three main spectral bands: ultraviolet radiation (UV, wavelength 290-400 nm), visible light (400-760 nm), and infrared radiation (IR, 760-4000 nm). Infrared radiation is subdivided into IRA (760-1440 nm), IRB (1440-3000 nm) and IRC (3000-4000 nm). It is well known that UV radiation can play an important role in premature skin ageing and can contribute to the development of skin cancer. Thus, protection against UV radiation is vitally important to the end-user. Titanium dioxide has long been used as an attenuator of UV radiation.

However, at the surface of the Earth, UV radiation only accounts for around 2% of total solar radiation. Infrared radiation accounts for over half of the total solar radiation at the Earth's surface, most of which being IRA radiation. It is also reported that IRA penetrates the skin deeper than visible light and UV radiation. Consequently, it is understood that IR radiation, particularly IRA radiation, can, like UV radiation, play an important role in skin damage, particularly premature skin aging.

This increased understanding of the significance and potential consequences of IR radiation is becoming more widely reported amongst scientific and consumer communities. Therefore, personal care products and cosmetics which can provide a degree of protection against IR radiation, particularly IRA radiation, are increasingly sought after by the end-user.

### SUMMARY OF THE INVENTION

It is an object of the present invention to address at least one of the above or other disadvantages associated with the prior art.

It has surprisingly been found that the use of titanium dioxide particles having particular properties and characteristics can, when present in a dispersion or slurry, provide beneficial attenuation of IR radiation.

Thus, according to a first aspect of the present invention, there is provided a dispersion of precursor titanium dioxide particles having an intensity mean peak value particle size, measured by dynamic light scattering (DLS), as herein described, or a number mean peak value particle size (measured by DLS as herein described) in the range from 0.2 to 2.0 μm within a dispersing medium, wherein the particle size distribution of the titanium dioxide particles in dispersion is narrower than the particle size distribution of the precursor titanium dioxide particles.

There is also provided the use of the dispersion of titanium dioxide particles of the first aspect of the invention to attenuate infrared radiation, particularly in a cosmetic composition, more particularly a cosmetic composition to provide protection against IR radiation, more particularly IRA radiation.

According to another aspect of the present invention, there is provided a personal care composition comprising the titanium dioxide dispersion of the first aspect of the invention.

Another aspect of the present invention provides a method for improving the infrared radiation reflecting properties of precursor titanium dioxide particles having an intensity mean peak value particle size (measured by DLS as herein described) or a number mean peak value particle size (measured by DLS as herein described) in the range from 0.2 to 2.0 μm, by preparing a dispersion of the precursor particles within a dispersing medium, wherein the distribution of particle sizes of the titanium dioxide particles in the dispersion is narrower than the distribution of particle sizes of the precursor titanium dioxide particles.

According to yet another aspect of the present invention, there is provided a method of preparing a titanium dioxide dispersion according to the first aspect of the invention, the said method involving:
a) mixing the precursor titanium dioxide particles with a dispersing medium; and
b) milling the mixture to provide the dispersion.

There is further provided the use of a titanium dioxide dispersion according to the first aspect of the invention as an SPF booster, or to boost the SPF, in a composition also comprising a UV absorber.

All of the features described herein may be combined with any of the above aspects, in any combination.

The present invention is based in part on the determination and recognition by the inventors that a composition of the first aspect of the invention has advantageous properties.

### DETAILED DESCRIPTION OF THE INVENTION

It will be understood that any upper or lower quantity or range limit used herein may be independently combined.

The precursor titanium dioxide particles according to the present invention may be prepared by standard procedures, such as using the chloride process, or by the sulphate process, or by the hydrolysis of an appropriate titanium compound such as titanium oxydichloride or an organic or inorganic titanate, or by oxidation of an oxidisable titanium compound, e.g. in the vapour state.

In one embodiment, the precursor titanium dioxide particles may be produced by treating titanium-containing ore such as ilmenite, rutile or anatase with sulphuric or hydrochloric acid to remove impurities, and then adding water or an oxidising agent to the resultant solution to precipitate hydrated TiO₂. Such a method is described in EP 1580166 B1, the contents of which are hereby incorporated by reference. Hydrated TiO₂ may be produced by hydrolysing a titanium alkoxide. Metatitanic acid produced as an intermediate of TiO₂ pigment in the commercial sulphuric acid process is a preferred starting material.

The precursor titanium dioxide particles may contain one or more additional metals or metal compounds. These metal compounds may be selected from aluminium, zinc and potassium compounds, or combinations thereof. In one embodiment, the precursor TiO₂ particles contain aluminium, zinc and potassium compounds, for example Al₂O₃, ZnO and K₂CO₃.

The precursor particles containing the metal compounds may be produced by any suitable method. Preferably, the precursor particles are produced according to the method described in EP 1580166 B1. The metal compounds may be any suitable compounds. Preferred metal compounds are described in EP 1580166 B1. The precursor TiO₂ particles preferably contain 0.1 to 0.5% by weight of an aluminium compound calculated as Al₂O₃, 0.2 to 1.0% by weight of zinc compound calculated as ZnO, and 0.1 to 0.5% by weight of a potassium compound calculated as K₂CO₃.

In an alternative embodiment, the precursor titanium dioxide particles may be doped with a dopant metal selected from the group consisting of aluminium, chromium, cobalt, copper, gallium, iron, lead, manganese, nickel, silver, tin, vanadium, zinc, zirconium, and combinations thereof. The dopant is preferably selected from the group consisting of chromium, cobalt, copper, iron, manganese, nickel, silver, and vanadium, more preferably from manganese and vanadium, particularly manganese, and especially in the 2+ and/or 3+ state.

Doping can be performed by normal methods known in the art. Doping is preferably achieved by co-precipitation of titanium dioxide and a soluble dopant complex such as manganese chloride or manganese acetate. Alternatively, doping can be performed by a baking technique by heating a titanium complex in the presence of a dopant complex, e.g. manganese nitrate, at a temperature of greater than 500° C. and normally up to 1,000° C.

Dopants can also be added by oxidizing a mixture containing a titanium complex and dopant complex, e.g. manganese acetate, such as by spraying the mixture through a spray atomizer into an oxidation chamber.

Doped titanium dioxide particles preferably comprise in the range from 0.01 to 3%, more preferably 0.05 to 2%, particularly 0.1 to 1%, and especially 0.5 to 0.7% by weight of dopant metal, preferably manganese, based on the weight of titanium dioxide.

The precursor titanium dioxide particles according to the present invention preferably comprise anatase and/or rutile crystal form. The titanium dioxide in the particles suitably comprises a major portion of rutile, preferably greater than 70%, more preferably greater than 80%, particularly greater than 90%, and especially greater than 95% and up to 100% by weight of rutile. In addition, the precursor titanium dioxide particles preferably comprise less than 10%, more preferably less than 5%, and particularly less than 2% by weight of amorphous titanium dioxide. The remaining titanium dioxide (i.e. up to 100%) is in crystalline form. In one embodiment, the titanium dioxide in the particles preferably is substantially all in crystalline form.

In one embodiment, the precursor titanium dioxide particles herein described are preferably calcined. The precursor titanium dioxide particles may be calcined at a temperature of greater than 600° C., suitably in the range from 700 to 1500° C., preferably 800 to 1400° C., more preferably 850 to 1200° C., and especially 900 to 1100° C.

The precursor titanium dioxide particles preferably have an intensity mean peak value particle size (measured by DLS according to the method as herein described) in the range from 0.3 to 1.7 μm, preferably from 0.4 to 1.4 μm, more preferably from 0.5 to 1.2 μm, desirably from 0.6 to 1.0 μm and especially from 0.7 to 0.9 μm.

The precursor titanium dioxide particles preferably have a number mean peak value particle size (measured by DLS according to the method as herein described) in the range from 0.3 to 1.7 μm, preferably from 0.4 to 1.4 μm, more preferably from 0.5 to 1.2 μm, desirably from 0.6 to 1.0 μm and especially from 0.7 to 0.9 μm.

The precursor titanium dioxide particles preferably have a BET specific surface area, measured as herein described, in the range from 0.1 to 10, suitably 0.5 to 8, preferably 1.0 to 6, more preferably 1.5 to 5, particularly 2.0 to 4, and especially 2.2 to 3.2 m² g⁻¹.

The precursor titanium dioxide particles preferably have
(i) an average pore diameter, measured as herein described by mercury porosimetry, in the range from 0.01 to 10, suitably 0.05 to 8, preferably 0.1 to 5, more preferably 0.2 to 4, particularly 0.3 to 3, and especially 10.4 to 1 μm; and/or
(ii) a total pore area at 59,950 psia, measured as herein described by mercury porosimetry, in the range from 0.1 to 20, suitably 0.5 to 15, preferably 1 to 10, more preferably 1.5 to 7, particularly 2 to 5, and especially 2.5 to 3.5 m² g⁻¹; and/or
(iii) a bulk density at 0.33 psia, measured as herein described by mercury porosimetry, in the range from 1.05 to 1.70 g/ml, preferably from 1.15 to 1.60 g/ml, more preferably from 1.25 to 1.50 g/ml and desirably from 1.35 to 1.40 g/ml; and/or
(iv) an apparent density at 59950 psia, measured as herein described by mercury porosimetry, in the range from 2.50 to 3.30 g/ml, preferably from 2.60 to 3.20 g/ml, more preferably from 2.70 to 3.10 g/ml, and desirably from 2.80 to 3.00 g/ml; and/or
(v) a porosity, measured as herein described by mercury porosimetry, in the range from 40 to 65%, preferably from 45 to 60%, more preferably from 47 to 57%, desirably from 49 to 55% and especially from 51 to 53%.

In one embodiment of the present invention, the precursor titanium dioxide particles according to the invention are coated with an inorganic and/or organic coating. Doped titanium dioxide particles may be uncoated, i.e. consist essentially of titanium dioxide and dopant.

When present, the inorganic coating is preferably an oxide of aluminium, zirconium or silicon, or mixtures thereof such as alumina and silica. The amount of inorganic coating, preferably alumina and/or silica, is suitably in the range from 1 to 15%, preferably 3 to 8%, more preferably 4 to 6%, particularly 4.5 to 5.5%, and especially 4.8 to 5.2% by weight, based on the weight of titanium dioxide core (or uncoated) particles.

In one embodiment of the invention, the precursor titanium dioxide particles are hydrophobic. The hydrophobicity of the precursor titanium dioxide can be determined by pressing a disc of titanium dioxide powder, and measuring the contact angle of a drop of water placed thereon, by standard techniques known in the art. The contact angle of a hydrophobic titanium dioxide is preferably greater than 50°.

The precursor titanium dioxide particles can be coated with a hydrophobizing agent in order to render them hydrophobic. Suitable coating materials are water-repellent, preferably organic, and include fatty acids, preferably fatty acids containing 10 to 20 carbon atoms, such as lauric acid, stearic acid and isostearic acid, salts of the above fatty acids such as sodium, potassium and/or aluminium salts, fatty alcohols, such as stearyl alcohol, and silicones such as polydimethylsiloxane and substituted polydimethylsiloxanes, and reactive silicones such as methylhydrosiloxane and polymers and copolymers thereof. Stearic acid and/or salt thereof is particularly preferred.

In one embodiment, the precursor titanium dioxide particles are treated with up to 15%, suitably in the range from 0.1 to 10%, preferably 0.5 to 7% and more preferably 1 to 5%, by weight of fatty acid, based on the weight of the titanium dioxide core particles.

In one embodiment, the coating layer comprises a silane coupling agent, preferably an organosilane, and more preferably of general Formula (1);

X₄₋ₙSi-[Lₘ-Y]ₙ (1)

wherein

Y is a functional group,

X is a hydrolysable group,

L is a linking group,

m is 0 or 1, preferably 1, and

n is 1 or 2, preferably 1.

Thus, a preferred silane coupling agent is of the general formula X₃—Si-L-Y.

The at least one functional group (Y) may be, for example, selected from the group consisting of methyl, ethyl, vinyl, carboxyl, glycidoxy, epoxy, glycidyl, amino, mercapto, acrylic, and methacrylic group. The functional group preferably comprises a nitrogen atom, and more preferably is an amine group. The amine group may be a primary, secondary, tertiary or quaternary group, and is preferably a primary amine group.

The preferred amine group is suitably of formula —NR₂, wherein each R individually is, or comprises, a group selected from the group consisting of hydrogen, lower (i.e. C1-C6) alkyl, aryl, lower alkylaryl, lower arylalkyl, alkenyl, cycloalkenyl, alkene, alkylene, arylene, alkylarylene, arylalkylene and cycloalkylene. In a preferred embodiment, each R is individually selected from the group consisting of hydrogen and a linear or branched C1-C6 alkyl group, more preferably hydrogen and a C1-C4 alkyl group, and particularly where both R groups are hydrogen.

The at least one hydrolysable group (X) may be —OR¹, —Cl, —Br, —I, and preferably is —OR¹, wherein each R¹ individually is, or comprises, a group selected from the group consisting of hydrogen, lower (i.e. C1-C6) alkyl, aryl, lower alkylaryl, lower arylalkyl, alkenyl, cycloalkenyl, alkene, alkylene, arylene, alkylarylene, arylalkylene and cycloalkylene. Preferably each R¹ is individually selected from the group consisting of hydrogen and a linear or branched C1-C6 alkyl group, more preferably a C1-C4 alkyl, particularly a C1-C2 alkyl group, and especially an ethyl group.

The optional linking group (L) may comprise or consist of an alkyl, aryl, alkylaryl, arylalkyl, cycloalkyl, alkenyl, cycloalkenyl, alkene, alkenylene, cycloalkenylene, alkylene, arylene, alkylarylene, arylalkylene, and/or cycloalkylene group. The linking group is preferably a linear or branched C1-C6 alkylene group, more preferably a C1-C4 alkylene group, and particularly a C3 alkylene, i.e. propyl, group.

Examples of suitable silane coupling agents include methyl trimethoxysilane, glycidoxypropyl trimethoxysilane, methacryloxypropyltri-methoxysilane, vinyl triethoxysilane, phenyl alkoxysilanes such as phenyl trialkoxysilane and diphenyl dialkoxysilane, dialkyl dialkoxysilanes such as dimethyl dimethoxysilane and dimethyl diethoxysilane, quaternary silanes, and amino silanes.

Amino silanes are preferred and suitable materials include aminoethyl trimethoxysilane, aminoethyl triethoxysilane, aminopropyl trimethoxysilane, aminopropyl triethoxysilane, methylaminopropyl trimethoxysilane, ethylaminopropyl trimethoxysilane, aminopropyl tripropoxysilane, aminoisobutyl trimethoxysilane, and aminobutyl triethoxysilane. An especially preferred amino silane is aminopropyl triethoxysilane (NH₂—CH₂CH₂CH₂—Si—[OCH₂CH₃]₃).

The amount of silane coupling agent, or reaction product thereof, present in the coating layer is suitably up to 15%, preferably in the range from 0.1 to 10%, more preferably 0.3 to 7%, and especially 0.5 to 3.5% by weight of based on the weight of the titanium dioxide core particles.

The silane coupling agent is suitably used in the coating layer in combination with an inorganic material and/or a fatty acid, both as defined herein. The inorganic material is suitably silica, is preferably amorphous, and more preferably is in a highly hydrated form, i.e. contains a high proportion of hydroxyl groups. The silica is preferably not in the form of dense silica. The fatty acid is preferably a stearic acid and/or salt thereof.

Suitably, titanium dioxide core particles are coated with inorganic material, preferably silica, are dispersed in water and heated to a temperature in the range from 50 to 80° C., after which the silane coupling agent is added which reacts with the surface of the inorganic material and/or the surface of the titanium dioxide core particles. The fatty acid and/or salt thereof is preferably applied after the inorganic material and the silane coupling agent.

The precursor titanium dioxide particles may be coated prior to, or after any calcination stage. In a preferred embodiment, any coating is applied to the particles after any calcination stage. Thus, it is preferred that uncoated precursor titanium dioxide particles are subjected to the calcination process herein described.

In one embodiment, the precursor titanium dioxide particles are coated in-situ, during the formation of a dispersion according to the present invention. Such coating may be applied by adding coating materials to the dispersion mixture before the milling process as herein described. Examples of materials which are suitable for the in-situ coating process are isostearic acid, oleth-3 phosphate, octyl/decyl phosphate, cetoleth-5 phosphate, PPG-5-ceteth-10 phosphate, trideceth-5 phosphate, dobanol C12-C15 phosphate, C9-C15 alkyl phosphate, glyceryl triacetate, sorbitan laurate, sorbitan isostearate, sodium lauryl sulfate, sodium methyl cocoyl taurate, and mixtures thereof.

The precursor titanium dioxide particles may be formed into a slurry, or preferably a liquid dispersion, in any suitable aqueous or organic liquid medium (“the dispersing medium”). By liquid is meant liquid at ambient temperature (e.g. at 25° C.), and by dispersion is meant a true dispersion, i.e. where the solid particles are stable to aggregation. The particles in the dispersion are relatively uniformly dispersed and resistant to settling out on standing, but if some settling out does occur, the particles can be easily re-dispersed by simple agitation.

Alternatively, the titanium dioxide particles may be in the form of a lotion or cream of a solid and/or semi-solid dispersion. Suitable solid or semi-solid dispersions may contain, for example, in the range from 50 to 90%, preferably 60 to 85% by weight of titanium dioxide particles, together with any one or more of the dispersing media disclosed herein, or a high molecular weight polymeric material, such as a wax, e.g. glyceryl monostearate.

For use in a cosmetic product, cosmetically acceptable materials are preferred as the dispersing medium. The dispersing medium may be water, or an organic medium such as a liquid, e.g. vegetable oil, fatty acid glyceride, fatty acid ester and/or fatty alcohol. One organic medium suitable as the dispersing medium is a siloxane fluid, especially a cyclic oligomeric dialkylsiloxane, such as the cyclic pentamer of dimethylsiloxane known as cyclomethicone. Alternative fluids suitable as the dispersing media include dimethylsiloxane linear oligomers or polymers having a suitable fluidity and phenyltris(trimethylsiloxy)silane (also known as phenyltrimethicone).

Examples of other organic media suitable for use as the dispersing medium include non-polar materials such as C13-C14 isoparaffin, isohexadecane, paraffinum liquidum (mineral oil), squalane, squalene, hydrogenated polyisobutene, and polydecene; and polar materials such as C12-C15 alkyl benzoate, caprylic/capric triglyceride, cetearyl isononanoate, ethylhexyl isostearate, ethylhexyl palmitate, isononyl isononanoate, isopropyl isostearate, isopropyl myristate, isostearyl isostearate, isostearyl neopentanoate, octyldodecanol, pentaerythrityl tetraisostearate, PPG-15 stearyl ether, triethylhexyl triglyceride, dicaprylyl carbonate, ethylhexyl stearate, Helianthus annus (sunflower) seed oil, isopropyl palmitate, and octyldodecyl neopentanoate, triethylhexanoin, ethylhexyl cocoate, propylene glycol isostearate, glyceryl isostearate, triisostearin, diethoxyethyl succinate, caprylyl eicosanoate, ethylhexyl hydroxystearate, lauryl lactate, butyl stearate, diisobutyl adipate, diisopropyl adipate, ethyl oleate, isocetyl stearate, propylene glycol dicaprylate/dicaprate, pentaerythrityl tetracaprylate/tetracaprate, oleyl oleate, propylene glycol isoceteth-3 acetate, PPG-3 benzyl ether myristate, cetearyl ethylhexanoate, ethylhexyl pelargonate, PPG-2 myristyl ether propionate, C14-18 alkyl ethylhexanoate, and mixtures thereof. The dispersing medium may comprise C12-C15 alkyl benzoate, caprylic/capric triglyceride, isostearyl isostearate, isopropyl isostearate, triisostearin, ethyl oleate or dicaprylyl ether, preferably the dispersing medium comprises C12-C15 alkyl benzoate, isostearyl isostearate or caprylic/capric triglyceride, more preferably comprises C12-C15 alkyl benzoate or isostearyl isostearate, especially comprises C12-C15 alkyl benzoate.

The dispersing medium may comprise a plant or vegetable oil, preferably the dispersing medium comprises sweet almond oil, olive oil, avocado oil, grapeseed oil, sunflower oil, meadowfoam seed oil, or carrot oil.

The dispersion according to the present invention may also contain a dispersing agent in order to improve the properties thereof. The dispersing agent is suitably present in the range from 0.01 to 15%, preferably 0.05 to 10%, more preferably 0.08 to 5%, and especially 0.1 to 3% by weight based on the total weight of titanium dioxide particles. It has surprisingly been found that the use of the precursor titanium dioxide particles of the present invention reduces the concentration of dispersing agent required to produce a true dispersion of the precursor particles in a dispersing medium when compared to the concentration required to prepare a true dispersion of smaller titanium dioxide particles.

Suitable dispersing agents include substituted carboxylic acids, soap bases and polyhydroxy acids. Typically the dispersing agent can be one having a formula R.CO.AX in which A is a divalent atom such as O, or a divalent bridging group. X can be hydrogen or a metal cation, or a primary, secondary or tertiary amino group or a salt thereof with an acid or a quaternary ammonium salt group. R may be the residue of a polyester chain which together with the —CO— group is derived from a hydroxy carboxylic acid of the formula HO—R′—COOH. As examples of typical dispersing agents are those based on ricinoleic acid, hydroxystearic acid, hydrogenated castor oil fatty acid which contains in addition to 12-hydroxystearic acid small amounts of stearic acid and palmitic acid. Dispersing agents based on one or more polyesters or salts of a hydroxycarboxylic acid and a carboxylic acid free of hydroxy groups can also be used. Compounds of various molecular weights can be used. Polyglyceryl-3 polyricinoleate and polyhydroxystearic acid are preferred dispersing agents. Polyglyceryl-3 polyricinoleate is particularly preferred when the coating layer of titanium dioxide particles comprises a silane coupling agent as herein defined. Polyhydroxystearic acid is particularly preferred when the coating layer of titanium dioxide particles does not comprise a silane coupling agent.

Other suitable dispersing agents are those monoesters of fatty acid alkanolamides and carboxylic acids and their salts. Suitable alkanolamides, for example, include those based on ethanolamine, propanolamine or aminoethyl ethanolamine. The dispersing agent can be one of those commercially referred to as a hyper dispersant. Polyhydroxystearic acid is a particularly preferred dispersing agent in organic media.

Suitable dispersing agents for use in an aqueous medium include a polymeric acrylic acid or a salt thereof. Partially or fully neutralized salts are usable e.g. the alkali metal salts and ammonium salts. Examples of dispersing agents are polyacrylic acids, substituted acrylic acid polymers, acrylic copolymers, sodium and/or ammonium salts of polyacrylic acids and sodium and/or ammonium salts of acrylic copolymers. Such dispersing agents are typified by polyacrylic acid itself and sodium or ammonium salts thereof as well as copolymers of an acrylic acid with other suitable monomers such as a sulphonic acid derivative such as 2-acrylamido 2-methyl propane sulphonic acid. Comonomers polymerisable with the acrylic or a substituted acrylic acid can also be one containing a carboxyl grouping. Usually the dispersing agents for use in an aqueous medium have a molecular weight in the range from 1,000 to 10,000 Da, and are preferably substantially linear molecules. Materials such as sodium citrate may also be used as a co-dispersant.

The dispersion according to the present invention may also contain a thickening agent in order to improve the properties thereof. The thickening agent is suitably present in the range from 0.01 to 10%, preferably 0.05 to 8%, more preferably 0.08 to 5%, particularly 0.1 to 3%, and especially 0.3 to 1% by weight based on the total weight of the dispersion.

Suitable thickening agents include Thixcin R, Fumed silica, Span 120, Arlacel 1690, Versathix, Procas H3, Bentone Gel TN, Cithrol DPHS, Bentone Gel IHD V, Hypermer B2465F, Hypermer KD14, Atlox 4912, Syncrowax ORM, Syncrowax OSW, Oleocraft LP-20, Oleocraft MP-30, Oleocraft HP-31, Oleocraft MP-32, Atlox 4916, Zephrym PD2206, Bentone 1000, Bentone SD-2, and Sucragel CF.

An advantage of the present invention is that dispersions, particularly liquid, can be produced which suitably contain at least 40%, preferably at least 50%, more preferably at least 60%, especially at least 65%, and generally up to 85%, by weight of precursor titanium dioxide particles based on the total weight of the dispersion.

In a preferred embodiment, the dispersion according to the present invention is prepared by the mixing together of the precursor titanium dioxide particles and the dispersing medium, stirring the mixture and milling the mixture.

Optionally, when a dispersing agent is used, the method of preparing the dispersion also comprises the step of adding the dispersing agent. When present, the dispersing agent is preferably added to the dispersing medium at the same time as the precursor titanium dioxide particles.

The mixture is preferably milled using a horizontal bead mill, preferably containing a grinding media. The mill is preferably operated at a rotational speed of between 2000 and 7000 r.p.m, more preferably between 3000 and 6000 r.p.m, and especially between 4000 and 5000 r.p.m.

The grinding media may be any suitable beads, for example, but not limited to glass, zirconia or steel beads, more preferably zirconia beads are used as the grinding media.

The size of the titanium dioxide particles in dispersion according to the present invention may be measured by techniques based on light scattering. The intensity of scattered light is measured, where this function is fit to obtain a size, using algorithms which determine (i) the cumulant (or Z-average) mean particle size, giving one overall average particle size, and (ii) the peak size which gives a mean size based on the intensity of the scattered light. Intensity values can also be used to give particle size distribution data, or can be converted to a number or volume distribution using Mie theory. This distribution describes the relative proportion of multiple components in the sample based on their mass or volume rather than based on their scattering (Intensity). In the present application, the light scattering measurements have been determined by Dynamic Light Scattering (DLS) according to the method described herein.

In one embodiment, the titanium dioxide particles in dispersion have an intensity based mean peak value particle size, as measured by DLS according to the method described herein, of (i) more than 400 nm, suitably more than 450 nm, more suitably more than 500 nm, preferably more than 550 nm, more preferably more than 600 nm, and especially more than 650 nm; and/or (ii) less than 1000 nm, suitably less than 950 nm, more suitably less than 900 nm, preferably less than 850 nm, more preferably less than 800 nm, and especially less than 750 nm; and/or (iii) any combination of (i) and (ii).

Preferably, the titanium dioxide particles in dispersion exhibit a smaller intensity based mean peak value particle size than that of the precursor titanium dioxide particles used to prepare the dispersion. Preferably, the titanium dioxide particles in dispersion exhibit, as measured by DLS according to the method described herein, at least a 2% smaller intensity based mean peak value particle size than the precursor titanium dioxide particles, preferably at least 3.5% smaller, preferably at least 5% smaller, more preferably at least 7.5% smaller, desirably at least 9% smaller and especially at least 12% smaller. The % difference is calculated by determining the % difference in measured intensity based mean peak value particle size, as measured by DLS according to the method described herein, between a titanium dioxide dispersion according to the invention and an analogous stirred suspension of precursor titanium dioxide particles, wherein the dispersion and suspension both comprise the same solids concentration of titanium dioxide.

In one embodiment the titanium dioxide particles in dispersion have an intensity based median particle size (the d50 particle size), measured by DLS as herein described, of (i) greater than 0.4 μm, suitably greater than 0.45 μm, more suitably greater than 0.50 μm, preferably greater than 55 μm, more preferably greater than 0.60 μm, particularly greater than 0.62 μm, and especially greater than 0.65 μm; and/or (ii) less than 0.73 μm, suitably less than 0.72 μm, more suitably less than 0.71 μm, preferably less than 0.70 μm, more preferably less than 0.69 μm, and especially less than 0.68 μm; and/or (iii) any combination of (i) and (ii).

Preferably, the titanium dioxide particles in dispersion exhibit a smaller intensity based d50 particle size than that of the precursor titanium dioxide particles used to prepare the dispersion. Preferably, the titanium dioxide particles in dispersion exhibit, as measured by DLS according to the method described herein, at least a 3.5% smaller intensity based d50 particle size than the precursor titanium dioxide particles, preferably at least 5% smaller, preferably at least 7.5% smaller, more preferably at least 10% smaller, and especially at least 13% smaller. The % difference is calculated by determining the % difference in measured intensity based d50 particle size, as measured by DLS according to the method described herein, between a titanium dioxide dispersion according to the invention and an analogous stirred suspension of precursor titanium dioxide particles, wherein the dispersion and suspension both comprise the same solids concentration of titanium dioxide.

The size distribution of the titanium dioxide particles in dispersion can be important. In a preferred embodiment, (i) less than 10% by number of titanium dioxide particles in dispersion have an intensity based particle size (the d10 particle size) of more than 275 nm, suitably more than 270 nm, more suitably more than 265 nm, preferably more than 260 nm, more preferably more than 255 nm, and especially more than 250 nm below the intensity based median particle size (d50); and/or (ii) more than 90% by number of titanium dioxide particles in dispersion have an intensity based diameter (the d90 particle size) of less than 455 nm, suitably less than 450 nm, more suitably less than 445 nm, preferably less than 440 nm, more preferably less than 435 nm, and especially less than 430 nm above the intensity based median particle size (d50); and/or (iii) any combination of (i) and (ii).

Preferably, the width of the intensity based particle size distribution (taken as the difference between the d90 particle size and d10 particle size) is from 200 to 730 nm, preferably from 250 to 720 nm, preferably from 250 to 710 nm, more preferably from 300 to 700 nm, desirably from 300 to 690 nm and especially from 400 to 680 nm.

Preferably, the titanium dioxide particles in dispersion exhibit a narrower intensity based particle size distribution than that of the precursor titanium dioxide particles used to prepare the dispersion. Preferably, the titanium dioxide particles in dispersion exhibit, as measured by DLS according to the method described herein and calculated as the difference between the intensity based d90 particle size and intensity based d10 particle size, at least a 1.5% narrower intensity based particle size distribution than the precursor titanium dioxide particles, preferably at least 2.8% narrower, preferably at least 4% narrower, more preferably at least 5.5% narrower, desirably at least 6.8% narrower and especially at least 8% narrower. The % difference is calculated by determining the % decrease in measured particle size distribution, as measured by DLS according to the method described herein, between a titanium dioxide dispersion according to the invention and an analogous stirred suspension of precursor titanium dioxide particles, wherein the dispersion and suspension both comprise the same solids concentration of titanium dioxide.

In one embodiment, the titanium dioxide particles in dispersion have a number based mean peak value particle size, as measured by DLS according to the method described herein, of (i) more than 350 nm, suitably more than 400 nm, more suitably more than 450 nm, preferably more than 500 nm, more preferably more than 550 nm, and especially more than 600 nm; and/or (ii) less than 900 nm, suitably less than 850 nm, more suitably less than 800 nm, preferably less than 750 nm, more preferably less than 700 nm, and especially less than 650 nm; and/or (iii) any combination of (i) and (ii).

Preferably, the titanium dioxide particles in dispersion exhibit a smaller number based mean peak value particle size than that of the precursor titanium dioxide particles used to prepare the dispersion. Preferably, the titanium dioxide particles in dispersion exhibit, as measured by DLS according to the method described herein, at least a 3% smaller number based mean peak value particle size than the precursor titanium dioxide particles, preferably at least 5% smaller, preferably at least 7% smaller, more preferably at least 10% smaller, desirably at least 13% smaller and especially at least 16% smaller. The % difference is calculated by determining the % difference in measured number based mean peak value particle size, as measured by DLS according to the method described herein, between a titanium dioxide dispersion according to the invention and an analogous stirred suspension of precursor titanium dioxide particles, wherein the dispersion and suspension both comprise the same solids concentration of titanium dioxide.

In one embodiment the titanium dioxide particles in dispersion have a number based median particle size (the d50 particle size), measured by DLS as herein described, of (i) greater than 0.30 μm, suitably greater than 0.35 μm, more suitably greater than 0.40 μm, preferably greater than 45 μm, more preferably greater than 0.50 μm, particularly greater than 0.53 μm, and especially greater than 0.56 μm; and/or (ii) less than 0.72 μm, suitably less than 0.70 μm, more suitably less than 0.67 μm, preferably less than 0.63 μm, more preferably less than 0.60 μm, and especially less than 0.58 μm; and/or (iii) any combination of (i) and (ii).

Preferably, the titanium dioxide particles in dispersion exhibit a smaller number based d50 particle size than that of the precursor titanium dioxide particles used to prepare the dispersion. Preferably, the titanium dioxide particles in dispersion exhibit, as measured by DLS according to the method described herein, at least a 5% smaller number based d50 particle size than the precursor titanium dioxide particles, preferably at least 7.5% smaller, preferably at least 10% smaller, more preferably at least 12.5% smaller, desirably at least 15% smaller and especially at least 18% smaller. The % difference is calculated by determining the % difference in measured number based d50 particle size, as measured by DLS according to the method described herein, between a titanium dioxide dispersion according to the invention and an analogous stirred suspension of precursor titanium dioxide particles, wherein the dispersion and suspension both comprise the same solids concentration of titanium dioxide.

The size distribution of the titanium dioxide particles in dispersion can be important. In a preferred embodiment, (i) less than 10% by number of titanium dioxide particles in dispersion have a number based particle size (the d10 particle size) of more than 275 nm, suitably more than 270 nm, more suitably more than 265 nm, preferably more than 260 nm, more preferably more than 255 nm, and especially more than 250 nm below the number based median particle size (d50); and/or (ii) more than 90% by number of titanium dioxide particles in dispersion have a number based diameter (the d90 particle size) of less than 455 nm, suitably less than 450 nm, more suitably less than 445 nm, preferably less than 440 nm, more preferably less than 435 nm, and especially less than 430 nm above the number based median particle size (d50); and/or (iii) any combination of (i) and (ii).

Preferably, the width of the number based particle size distribution (taken as the difference between the d90 particle size and d10 particle size) is from 200 to 770 nm, preferably from 250 to 760 nm, preferably from 250 to 745 nm, more preferably from 300 to 730 nm, desirably from 300 to 715 nm and especially from 400 to 700 nm.

Preferably, the titanium dioxide particles in dispersion exhibit a narrower number based particle size distribution than that of the precursor titanium dioxide particles used to prepare the dispersion. Preferably, the titanium dioxide particles in dispersion exhibit, as measured by DLS according to the method described herein and calculated as the difference between the number based d90 particle size and number based d10 particle size, at least a 2% narrower number based particle size distribution than the precursor titanium dioxide particles, preferably at least 3.5% narrower, preferably at least 5% narrower, more preferably at least 7% narrower, more preferably at least 8.5% smaller, desirably at least 10% narrower and especially at least 12% narrower. The % difference is calculated by determining the % decrease in measured particle size distribution, as measured by DLS according to the method described herein, between a titanium dioxide dispersion according to the invention and an analogous stirred suspension of precursor titanium dioxide particles, wherein the dispersion and suspension both comprise the same solids concentration of titanium dioxide.

The titanium dioxide dispersion according to the present invention preferably exhibits an acceptable transparency, and may have an extinction coefficient at 524 nm (E₅₂₄), measured as herein described, of (i) less than or equal to 5.0, suitably less than or equal to 4.0, more preferably less than or equal to 3.2, and especially less than or equal to 2.6 l/g/cm; and/or (ii) greater than or equal to 1.5, suitably greater than or equal to 1.8, more preferably greater than or equal to 2.0, and especially greater than or equal to 2.2 l/g/cm; and/or (iii) any combination of (i) and (ii).

The titanium dioxide dispersion of the present invention preferably displays, when measured as herein described by a N-IR spectrophotometer:
(i) a reflectance of infrared radiation of wavelength 1000 nm of at least 30%, preferably at least 35%, more preferably at least 40% and especially at least 45%, and generally up to 80%; and/or
(ii) a reflectance of infrared radiation of wavelength 1200 nm of at least 25%, preferably at least 30%, more preferably at least 35% and especially at least 40%, and generally up to 80%; and/or
(iii) a reflectance of infrared radiation of wavelength 1400 nm of at least 25%, preferably at least 30%, more preferably at least 35% and especially at least 40%, and generally up to 80%.

The titanium dioxide dispersion preferably provides an increase in reflectance of infrared radiation, when measured as herein described by a N-IR spectrophotometer, compared to that of the precursor titanium dioxide particles, of:
(i) at least 2%, preferably at least 4%, more preferably at least 6%, desirably at least 8% and especially at least 10% at a wavelength of 1000 nm; and/or
(ii) at least 2.5%, preferably at least 4.5%, more preferably at least 7%, desirably at least 10% and especially at least 13% at a wavelength of 1200 nm; and/or
(iii) at least 2%, preferably at least 4.5%, more preferably at least 7%, desirably at least 10% and especially at least 12% at a wavelength of 1400 nm.

The % difference in IR reflectance is calculated by determining the % increase in measured IR reflectance at each wavelength between a titanium dioxide dispersion according to the invention and an analogous stirred suspension of precursor titanium dioxide particles, wherein the dispersion and suspension both comprise the same solids concentration of titanium dioxide.

The titanium dioxide dispersion of the present invention preferably prevents, as measured using a Hydrosun Infrared Lamp according to the method as herein described, transmission of at least 10% of infrared radiation between the wavelengths of 700 nm and 1400 nm, preferably at least 15%, more preferably at least 20%, and especially at least 25% and generally up to 80%. By “prevention of transmission”, it is meant prevention of radiation travelling through the sample by any means including reflectance and/or absorbance.

The titanium dioxide dispersion of the present invention preferably prevents, as measured using the Hydrosun Infrared Lamp according to the method as herein described, transmission of at least 25% more IR radiation between the wavelengths of 780 nm and 1400 nm compared to the precursor titanium dioxide particles, preferably at least 35% more, more preferably at least 45% more, and desirably at least 55% more. The difference in IR transmission is calculated by determining the % increase in measured IR transmission between a titanium dioxide dispersion according to the invention and an analogous stirred suspension of precursor titanium dioxide particles, wherein the dispersion and suspension both comprise the same solids concentration of titanium dioxide.

In one embodiment, a composition, preferably personal care composition, more preferably an end-use skin or hair care composition, containing the titanium dioxide dispersion according to the present invention preferably comprises greater than 0.1%, more preferably in the range from 0.1 to 20%, particularly 0.5 to 15%, and especially 1 to 10% by weight based on the total weight of the composition of titanium dioxide solids in dispersion.

The titanium dioxide dispersions of the present invention are useful as ingredients for preparing cosmetic compositions, especially in the form of oil-in-water or water-in-oil emulsions. The compositions may further contain conventional additives suitable for use in the intended application, such as conventional cosmetic ingredients used in sunscreens, makeup, skin care and hair care compositions. The titanium dioxide dispersions of the present invention may be the only infrared radiation attenuator present in the cosmetic composition. Alternatively, other infrared radiation attenuators may be present in the composition.

In one embodiment, one or more sunscreen agents or UV absorbers, such as other titanium dioxide, zinc oxide and/or other organic UV absorbers may also be added to provide a full spectrum solar radiation, or a UV and IR, attenuating composition. For example, the titanium dioxide dispersions defined herein may be used in combination with other existing commercially available titanium dioxide and/or zinc oxide sunscreens.

The titanium dioxide dispersions of the present invention may be used in combination with organic UV absorbers such as butyl methoxydibenzoylmethane (avobenzone), benzophenone-3 (oxybenzone), 4-methylbenzylidene camphor (enzacamene), benzophenone-4 (sulisobenzone), bis-ethylhexyloxyphenol methoxyphenyl triazine (bemotrizinol), diethylamino hydroxybenzoyl hexyl benzoate, diethylhexyl butamido triazone, disodium phenyl dibenzimidazole tetrasulfonate, drometrizole trisiloxane, ethylhexyl dimethyl PABA (padimate O), ethylhexyl methoxycinnamate (octinoxate), ethylhexyl salicylate (octisalate), ethylhexyl triazone, homosalate, isoamyl p-methoxycinnamate (amiloxate), isopropyl methoxycinnamate, menthyl anthranilate (meradimate), methylene bis-benzotriazolyl tetramethylbutylphenol (bisoctrizole), octocrylene, PABA (aminobenzoic acid), phenylbenzimidazole sulfonic acid (ensulizole), terephthalylidene dicamphor sulfonic acid, and mixtures thereof.

When used, the UV attenuators or sunscreens are preferably present at a concentration in the range from 0.5% to 25%, more preferably 1 to 20%, and particularly 2 to 15% by weight of the total cosmetic composition. Preferably, when present, the UV attenuator is present at a higher concentration that the titanium dioxide dispersion of the present invention.

The titanium dioxide dispersions of the present invention may suitably be used as an SPF booster in a sunscreen composition when present in combination with a UV absorber. It has surprisingly been found that the titanium dioxide dispersions of the present invention can improve the SPF effect of UV absorbers.

Preferably, the titanium dioxide particles of the present invention exhibit an SPF boosting effect in a sunscreen composition with a UV absorber of at least 5%, preferably at least 10%, preferably at least 15%, preferably at least 18%, desirably at least 25%, and up to 80% based on the measured SPF of the composition without the titanium dioxide dispersion present. The SPF boosting effect is calculated by determining the % increase in measured SPF between a sunscreen composition comprising a UV absorber and a titanium dioxide dispersion according to the invention and an analogous sunscreen composition without the titanium dioxide dispersion according to the invention.

It has also advantageously been discovered that, when present, a UV attenuator does not adversely affect the performance of the titanium dioxide dispersion of the present invention in terms of N-IR reflectance or prevention of transmission of IR radiation of wavelengths between 780 and 1400 nm

In the cosmetic compositions of the invention, the oil phase of the emulsion may comprise at least one ester oil, vegetable oil, alcohol, paraffin oil or silicone.

Suitable oil phase components include non-polar oils, for example mineral or paraffin, especially isoparaffin, oils, such as that sold by Croda as Arlamol™ HD; or medium polarity oils, for example vegetable ester oils such as jojoba oil, vegetable glyceride oils, animal glyceride oils, such as that sold by Croda as Crodamol™ GTCC (caprylic/capric triglyceride), synthetic oils, for example synthetic ester oils, such as isopropyl palmitate and those sold by Croda as Crodamol IPP and Arlamol DOA, ether oils, particularly of two fatty e.g. C8 to C18 alkyl residues, such as that sold by BASF as Cetiol OE (dicaprylether), guerbet alcohols such as that sold by BASF as Eutanol G (octyl dodecanol), or silicone oils, such as dimethicone oil such as those sold by Dow Corning as Xiameter PMX-200, cyclomethicone oil, or silicones having polyoxyalkylene side chains to improve their hydrophilicity; or highly polar oils including alkoxylate emollients for example fatty alcohol propoxylates such as that sold by Croda as Arlamol PS15E (propoxylated stearyl alcohol). Suitable emollient materials that can be solid at ambient temperature but liquid at temperatures typically used to make the compositions of this invention include jojoba wax, tallow and coconut wax/oil. When non-polar oils are used it may be desirable to use relatively high concentrations of the composition according to the present invention, in order to achieve suitably satisfactory emulsification, particularly to obtain small oil droplets.

Mixtures of emollients can and often will be used, and in some cases solid emollients may dissolve wholly or partly in liquid emollients or in combination the freezing point of the mixture is suitably low. Where the emollient composition is a solid (such as fatty alcohols) at ambient temperature, the resulting dispersion may technically not be an emulsion (although in most cases the precise phase of the oily disperse phase cannot readily be determined) but such dispersions behave as if they were true emulsions and the term emulsion is used herein to include such compositions.

The concentration of the oil phase may vary widely. The amount of oil in the emulsion is suitably in the range from 1 to 90%, preferably 3 to 60%, more preferably 5 to 40%, particularly 8 to 20%, and especially 10 to 15% by weight of the total formulation.

The amount of water (or polyol, e.g. glycerin) present in the emulsion is suitably greater than 5%, preferably in the range from 30 to 90%, more preferably 50 to 90%, particularly 70 to 85%, and especially 75 to 80% by weight of the total formulation.

Cosmetic compositions according to the present invention may also contain surfactant materials which form part of the emulsifier system. Suitable surfactants include relatively hydrophilic surfactants, e.g. having a HLB value of greater than 10, preferably greater than 12, and relatively hydrophobic surfactants e.g. having a HLB value of less than 10, preferably less than 8. Relatively hydrophilic surfactants include alkoxylate surfactants with an average in the range from about 10 to about 100 alkylene oxide, particularly ethylene oxide residues; and relatively hydrophobic surfactants include alkoxylate surfactants preferably with an average in the range from about 3 to about 10 alkylene oxide, particularly ethylene oxide residues.

Many other components may be included in the cosmetic composition. These components can be oil soluble, water soluble or non-soluble. Examples of such materials include:
(i) preservatives such as those based on potassium sorbate, sodium benzoate, parabens (alkyl esters of 4-hydroxybenzoic acid), phenoxyethanol, substituted ureas and hydantoin derivatives e.g. those sold commercially under the trade names Germaben II Nipaguard BPX and Nipaguard DMDMH. Such preservatives are used preferably at a concentration in the range from 0.5 to 2% by weight of the total composition. A preservative booster such as caprylyl glycol may also be used;
(ii) perfumes, when used preferably at a concentration in the range from 0.1 to 10% more preferably up to about 5%, and particularly up to about 2% by weight of the total composition;
(iii) humectants or solvents such as alcohols, polyols such as glycerol and polyethylene glycols, when used preferably at a concentration in the range from 1 to 10% by weight of the total composition;
(iv) alpha hydroxy acids such as glycolic, citric, lactic, malic, tartaric acids and their esters; self-tanning agents such as dihydroxyacetone;
(v) antimicrobial, particularly anti-acne components such as salicylic acid;
(vi) vitamins and their precursors including: (a) Vitamin A, e.g. as retinyl palmitate and other tretinoin precursor molecules, (b) Vitamin B, e.g. as panthenol and its derivatives, (c) Vitamin C, e.g. as ascorbic acid and its derivatives, (d) Vitamin E, e.g. as tocopheryl acetate, (e) Vitamin F, e.g. as polyunsaturated fatty acid esters such as gamma-linolenic acid esters;
(vii) skin care agents such as ceramides either as natural materials or functional mimics of natural ceramides;
(vii) phospholipids, such as synthetic phospholipids or natural phospholipids, e.g. lecithin;
(ix) vesicle-containing formulations;
(x) germanium-containing compounds;
(xi) botanical extracts with beneficial skin care properties;
(xii) skin whiteners such as Arlatone Dioic DCA (trade mark) sold by Croda, kojic acid, arbutin and similar materials;
(xiii) skin repair compounds actives such as Allantoin and similar series;
(xiv) caffeine and similar compounds;
(xv) cooling additives such as menthol or camphor;
(xvi) insect repellents such as N,N-diethyl-3-methylbenzamide (DEET) and citrus or eucalyptus oils;
(xvii) essential oils;
(xviii) ethanol;
(xix) pigments, including microfine pigments, particularly oxides and silicates, e.g. iron oxide, particularly coated iron oxides, and/or titanium dioxide, and ceramic materials such as boron nitride;
(xx) other solid components, such as are used in make up and cosmetics, to give suspoemulsions, preferably used in an amount in the range from 1 to 15 wt %, more preferably from 5 to 15 wt % based on the total weight of the composition; and (xxi) deodorant or antiperspirant agents, for example aluminium salts such as aluminium chlorohydrate. Such agents are typically present in a formulation at a concentration of up to 40% by weight (solids) based on the total weight of the composition, preferably in the range from 1 to 40 wt % (solids), more preferably from 10 to 25 wt % (solids) based on the total weight of the composition;
(xxii) depilatory agents such as potassium thioglycollate or calcium thioglycollate.

Such agents are typically present in a composition at a concentration of up to 15% by weight (active) based on the total weight of the composition, preferably between in the range from 1 to 15 wt % (active), more preferably from 2 to 7 wt % (active) based on the total weight of the composition;
(xxiii) hair relaxing agents such as potassium hydroxide or sodium hydroxide.

Such agents are typically present in a composition at a concentration of up to 15% by weight based on the total weight of the composition, preferably between in the range from 0.1 to 10 wt %, more preferably from 0.5 to 5 wt % (solids) based on the total weight of the composition.

The cosmetic compositions and emulsions according to the present invention are suitable for use in a wide range of formulations and end-use applications, such as moisturizers, sunscreens, after sun products, body butters, gel creams, high perfume containing products, perfume creams, baby care products, hair treatments, hair conditioners, skin toning and skin whitening products, water-free products, anti-perspirant and deodorant products, tanning products, cleansers, 2-in-1 foaming emulsions, multiple emulsions, preservative free products, mild formulations, scrub formulations e.g. containing solid beads, silicone in water formulations, pigment containing products, sprayable emulsions, cosmetics, colour cosmetics, shower products, foaming emulsions, make-up remover, eye make-up remover, and wipes.

It is to be understood that none of the above-described features are mutually exclusive. Any of the above features may be taken in any combination and with any aspect of the invention.

Test Methods

It will be understood that all tests and physical properties listed have been determined at atmospheric pressure and ambient temperature (i.e. about 23° C.), unless otherwise stated herein, or unless otherwise stated in the referenced test methods and procedures.

In this specification the following test methods have been used:

1) BET Specific Surface Area Measurement

A sample of precursor titanium dioxide powder was assessed using a Micromeritics Gemini VII 2390P. 0.4-0.5 g of dry titanium dioxide powder was introduced into sample tubes, degassed for 10 minutes under nitrogen at room temperature, before being heated to 200° C. and held at this temperature for 3 hours, again under nitrogen. The dry sample was immersed in liquid nitrogen (−196° C.) and once the sample was frozen, the specific surface area (SSA) was analysed using nitrogen.

2) Mercury Porosimetry

The pore size distribution of the samples was measured using a Micromeritics Autopore V Porosimeter. Approximately 0.28 g of powder of the precursor titanium dioxide particles was weighed into the bulb of the penetrometer. The penetrometer containing the sample was loaded into the Micromeritics Autopore V porosimeter and measurements were carried out between 0.1 to 61,000 psia during intrusion and extrusion cycles. The average pore diameter, total pore area, bulk density, apparent density and porosity were determined.

3) DLS Measurements

Samples for the DLS were prepared by dilution (˜0.1% or ˜0.05 g in 50 g) of each of the dispersion of Example 1 and the suspension of Comparative Example A in a 3% Hypermer LP1 in Crodamol AB (C12-C15 alkyl benzoate) mixture. The samples were ultrasonicated for 15 minutes, transferred to a disposable plastic cuvette and analysed on a Malvern Zetasizer Nano ZS. The instrument initially started by measuring an equilibrium stage, followed by analyzing the scattered light intensity from the sample, determining hydrodynamic volume of the particles based on their Brownian motion in suspension. The cumulant mean (Z-average) value was calculated by the methods of cumulants described in, e.g. Koppel, D. E. “Analysis of Macromolecular Polydispersity in Intensity Correlation Spectroscopy: The Method of Cumulants” J. Chem. Phys 57 (11), pp 4814-4820, 1972 and the mean peak value particle size was also determined. The intensity based d10, d50 and d90 particle sizes and number based d10, d50 and d90 particle sizes were also determined.

4) Extinction Coefficients

0.1 g sample of the dispersion of Example 1 was diluted with 100 ml of cyclohexane. This diluted sample was then further diluted with cyclohexane in the ratio sample:cyclohexane of 1:19. The total dilution was 1:20,000.

The diluted sample was placed in a spectrophotometer (Perkin-Elmer Lambda 650 UV/VIS Spectrophotometer) with a 1 cm path length and the absorbance of UV and visible light measured. Extinction coefficients were calculated from the equation A=E.c.I, where A=absorbance, E=extinction coefficient in litres per gram per cm, c=concentration in grams per litre, and I=path length in cm. A focus was placed on E₅₂₄ to give an indication of the transparency of the sample.

5) N-IR Reflectance

0.5 g each of the dispersion of Example 1 and the suspension of Comparative Example A were mixed with 24.5 g of Crodamol AB to make 1.4% solids solutions. These solutions were pipetted into a 7 ml vial and left for 2 hours. The vials were shaken and run on a N-IR DRIFTS spectrometer (Nicolet iS5N, Thermo Fisher Scientific). The % of IR radiation reflected at various wavelengths was determined. The method was repeated twice more, shaking the vials before each measurement was taken, and an average value determined.

6) Total IR-A Transmission

2 mg/cm² of each of the dispersion of Example 3 and the suspension of Comparative Example B were added to transpore tape. The tape was stuck to a PMMA plate and the transmittance of infrared radiation of wavelengths between 780 and 1400 nm (generated using a Hydrosun InfraRed (IR) lamp at 100 mW/cm2) through the transpore tape was measured for each sample using a detector (ILT1400 radiometer). The percentages of IR—The % of radiation detected compared to a control sample (using transpore tape only) was measured. A reduction in value would indicate the sample is demonstrating reflection or absorption properties.

7) Sun Protection Factor Measurements

The Sun Protection Factor (SPF) of a sunscreen formulation (e.g. as described in Example 7) was determined using the in vitro method of Diffey and Robson, J. Soc. Cosmet. Chem. Vol. 40, pp 127-133, 1989, employing a spreadmaster robot.

### EXAMPLES

The invention will now be illustrated, by way of example only, in the following Examples. All parts and percentages are given by weight unless otherwise stated.

### Example 1—Preparation of Dispersion of Titanium Dioxide Particles

An organic liquid dispersion of titanium dioxide particles was produced by mixing 0.12 g of polyhydroxystearic acid with 29.88 g of C12-C15 alkylbenzoate, and then adding 70 g of precursor titanium dioxide powder (MPY-100EX ex Tayca) into the mixture. The mixture was stirred using an overhead stirrer at 800 rpm, for 15 minutes and then passed through a horizontal bead mill, operating at 4,500 r.p.m. and containing zirconia beads as grinding media, for 15 minutes. The result was a true dispersion of titanium dioxide within the dispersing medium.

### Example 2—Preparation of Dispersion of Titanium Dioxide Particles Including Thickening Agent

An organic liquid dispersion of titanium dioxide particles was produced by mixing 0.12 g of polyhydroxystearic acid with 29.355 g of C12-C15 alkylbenzoate and 0.525 g of trihydroxystearin (Thixcin R ex Elementis) and then adding 70 g of precursor titanium dioxide powder (MPY-100EX ex Tayca) into the mixture. The mixture was stirred using an overhead stirrer at 800 rpm, for 15 minutes and then passed through a horizontal bead mill, operating at 4,500 r.p.m. and containing zirconia beads as grinding media, for 15 minutes. The result was a true dispersion of titanium dioxide within the dispersing medium.

### Example 3—Preparation of Dispersion of Titanium Dioxide Particles

The dispersion of Example 1 was further diluted to 2.5% solids using a solution of C12-C15 alkylbenzoate containing 0.12% polyhydroxystearic acid. The resulting dispersion was mixed using an overhead stirrer at 800 rpm, for 15 minutes.

### Comparative Example A—Preparation of Suspension of Precursor Titanium Dioxide Particles

An organic liquid suspension of titanium dioxide particles was produced by mixing 0.12 g of polyhydroxystearic acid with 29.88 g of C12-C15 alkylbenzoate, and then adding 70 g of precursor titanium dioxide powder (MPY-100EX ex Tayca) into the mixture. The mixture was stirred using an overhead stirrer at 800 rpm, for 15 minutes. The result was a suspension of precursor titanium dioxide particles in the organic liquid medium.

### Comparative Example B—Preparation of Suspension of Precursor Titanium Dioxide Particles

The suspension of Comparative Example A was further diluted to 2.5% solids using a solution of C12-C15 alkylbenzoate containing 0.12% polyhydroxystearic acid. The resulting suspension was mixed using an overhead stirrer at 800 rpm, for 15 minutes.

### Example 4—Analysis of Dispersion and Suspension

The Examples and Comparative Examples described above were analysed according to the test methods described herein and the following results observed.

i) DLS Particle Size Measurement

TABLE 1
Intensity-based particle sizes

| | | Comparative |
|---|---|---|
| | Example 1 (nm) | Example A (nm) |
| d10 | 420 | 489 |
| d50 | 670 | 774 |
| d90 | 1100 | 1230 |
| Difference between | 680 | 741 |
| d10 and d90 | | |
| Mean peak value | 717.3 | 819.3 |

TABLE 2
Number-based particle sizes

| | | Comparative |
|---|---|---|
| | Example 1 (nm) | Example A (nm) |
| d10 | 341 | 432 |
| d50 | 578 | 723 |
| d90 | 1030 | 1220 |
| Difference between | 689 | 788 |
| d10 and d90 | | |
| Mean peak value | 637.2 | 776.6 |

ii)

TABLE 3
N-IR reflectance

| IR radiation | | Comparative |
|---|---|---|
| wavelength (nm) | Example 1 | Example A |
| 1000 | 47.47% | 43.13% |
| 1200 | 40.86% | 36.05% |
| 1400 | 40.69% | 36.24% |

iii)

TABLE 4
Prevention of IR transmission

| | | % IR | |
|---|---|---|---|
| | % IR | blocked | |
| | transmission | (between | |
| | (between | 780 nm | |
| | 780 nm and | and | Standard |
| | 1400 nm) | 1400 nm) | Dev |
| Control (Transpore | 100 | 0 | 0 |
| tape only) | | | |
| Comparative | 82.7 | 17.3 | 1.484924 |
| Example B | | | |
| Example 3 | 71.4 | 28.6 | 1.555635 |

### Example 5—Combination of Dispersion with UV Attenuators

The formulations in Table 5 were prepared according to the following method.

The Keltrol, Veegum and Pricerine were pre-mixed, and then adding the water with stirring. The mixture was heated to 75-85° C. The Part A components were then combined and heated to 75-85° C. before adding Part B to Part A. The resulting mixture was briefly reheated. The Part AB mixture was stirred to ensure the oils were mixed, and was then added to Part C with stirring. The mixture was homogenised at 10000 rpm for 1 minute per 100 g, and then cooled to room temperature while stirring. Part D was then added with gentle stirring.

The percentage of IR-A radiation stopped, ie reflected or adsorbed, by each of the formulations were measured according to the method outlined by Elise Delamour et al (HelioScreen Labs, Creil, France) in Cosmetics & Toiletries®, Vol. 132, No. 9, October 2017, pages 45 to 58, and the results set out in Table 6 obtained.

TABLE 5
Formulations for Example 5

| | Formulation | Comparative | Formulation |
|---|---|---|---|
| | 01 | formulation X | 02 |
| Ingredient | % w/w | % w/w | % w/w |
| PART A | | | |
| Arlacel 165 | 3.00 | 3.00 | 3.00 |
| Span 60 | 3.00 | 3.00 | 3.00 |
| Tween 60 | 0.40 | 0.40 | 0.40 |
| Crodacol S95 | 0.50 | 0.50 | 0.50 |
| Crodamol AB | 15.75 | 15.75 | 15.75 |
| Crodamol SSA | 8.00 | 8.00 | 8.00 |
| Xiameter PMX-200 | 2.00 | 2.00 | 2.00 |
| silicone fluid 350 cs | | | |
| PART B | | | |
| Solaveil XT-100 | 0.00 | 9.09 | 9.09 |
| Solaveil CT-100 | 0.00 | 11.11 | 11.11 |
| Dispersion of Example 1 | 7.14 | 0.00 | 7.14 |
| PART C | | | |
| Water | 55.21 | 42.15 | 35.01 |
| Pricerine 9091 | 3.00 | 3.00 | 3.00 |
| Veegum Ultra | 0.80 | 0.80 | 0.80 |
| Keltrol CG-SFT | 0.20 | 0.20 | 0.20 |
| PART D | | | |
| Euxyl K350 | 1.00 | 1.00 | 1.00 |

TABLE 6
% of IR radiation prevented from transmission

| Formulations | % IRA | % IRB |
|---|---|---|
| Formulation 01 | 19.8 | 13.3 |
| Comparative Formulation X | 9.1 | 4.0 |
| Formulation 02 | 28.5 | 18.6 |

The results show that the combination of the dispersions according to the invention with UV attenuators, does not impede the performance of the dispersion in terms of prevention of IR radiation transmission.

### Example 6—Formulations

TABLE 7
Formulations for Example 6

| | Formulation 6A | | Formulation 6B | | Formulation 6C | |
|---|---|---|---|---|---|---|
| | | Weight | | Weight | | Weight |
| Ingredient | % w/w | (g) | % w/w | (g) | % w/w | (g) |
| PART A | | | | | | |
| Arlacel 165 | 3.00 | 9.00 | 3.00 | 9.00 | 3.00 | 9.00 |
| Span 60 | 3.00 | 9.00 | 3.00 | 9.00 | 3.00 | 9.00 |
| Tween 60 | 0.40 | 1.20 | 0.40 | 1.20 | 0.40 | 1.20 |
| Crodacol S95 | 0.50 | 1.50 | 0.50 | 1.50 | 0.50 | 1.50 |
| Crodamol AB | 15.75 | 47.25 | 15.75 | 47.25 | 15.75 | 47.25 |
| Crodamol SSA | 8.00 | 24.00 | 8.00 | 24.00 | 8.00 | 24.00 |
| Xiameter PMX-200 | | | | | | |
| silicone fluid 350 cs | 2.00 | 6.00 | 2.00 | 6.00 | 2.00 | 6.00 |
| Eusolex OCR | | | | | 4.50 | 13.50 |
| Eusolex OS | | | | | 2.50 | 7.50 |
| Eusolex 9020 | | | | | 2.00 | 6.00 |
| PART B | | | | | | |
| Solaveil XT-100 | 9.09 | 27.27 | | | | |
| Solaveil CT-100 | | | 11.11 | 33.33 | | |
| Dispersion of Example 1 | 7.14 | 21.42 | 7.14 | 21.42 | 7.14 | 21.42 |
| Solaveil CT-12W | | | | | 11.00 | 33.00 |
| PART C | | | | | | |
| Water | 46.12 | 138.36 | 44.10 | 132.30 | 35.21 | 105.63 |
| Pricerine 9091 | 3.00 | 9.00 | 3.00 | 9.00 | 3.00 | 9.00 |
| Veegum Ultra | 0.80 | 2.40 | 0.80 | 2.40 | 0.80 | 2.40 |
| Keltrol CG-SFT | 0.20 | 0.60 | 0.20 | 0.60 | 0.20 | 0.60 |
| PART D | | | | | | |
| Euxyl K350 | 1.00 | 3.00 | 1.00 | 3.00 | 1.00 | 3.00 |
| | 100.00% | 300.00 g | 100.00% | 300.00 g | 100.00% | 300.00 g |

| | Formulation 6D | | Formulation 6E | |
|---|---|---|---|---|
| | | Weight | | Weight |
| Ingredient | % w/w | (g) | % w/w | (g) |
| PART A | | | | |
| Arlacel 165 | 3.00 | 9.00 | 3.00 | 9.00 |
| Span 60 | 3.00 | 9.00 | 3.00 | 9.00 |
| Tween 60 | 0.40 | 1.20 | 0.40 | 1.20 |
| Crodacol S95 | 0.50 | 1.50 | 0.50 | 1.50 |
| Crodamol AB | 15.75 | 47.25 | 15.75 | 47.25 |
| Crodamol SSA | 8.00 | 24.00 | 8.00 | 24.00 |
| Xiameter PMX-200 | | | | |
| silicone fluid 350 cs | 2.00 | 6.00 | 2.00 | 6.00 |
| PART B | | | | |
| Solaveil XT-100 | | | 9.09 | 27.27 |
| Solaveil CT-100 | | | 11.11 | 33.33 |
| Dispersion of Example 1 | 7.14 | 21.42 | 7.14 | 21.42 |
| PART C | | | | |
| Water | 55.21 | 165.63 | 35.01 | 105.03 |
| Pricerine 9091 | 3.00 | 9.00 | 3.00 | 9.00 |
| Veegum Ultra | 0.80 | 2.40 | 0.80 | 2.40 |
| Keltrol CG-SFT | 0.20 | 0.60 | 0.20 | 0.60 |
| PART D | | | | |
| Euxyl K350 | 1.00 | 3.00 | 1.00 | 3.00 |
| | 100.00% | 300.00 g | 100.00% | 300.00 g |

Procedure:

The Keltrol, Veegum and Pricerine were premixed and then the water was added with stirring. The mixture was heated to 75-80° C. In a separate vessel, Part A was combined and heated to 75-80° C. Part B was added to Part A and the mixture briefly reheated. Parts AB and C were then added together with stirring. The resulting mixture was homogenised at 10000 rpm for 1 minute per 100 g, and then cooled to room temperature under stirring. Finally, Part D was added with gentle stirring.

### Example 7—Effect of Dispersion on In-Vitro SPF in Combination with UV Absorbers

The formulations in Table 8 were prepared according to the following method. The Keltrol, Veegum and Pricerine were premixed and then the water was added with stirring. The mixture was heated to 75-80° C. In a separate vessel, Part A was combined and heated to 75-80° C. Part B was added to Part A and the mixture briefly reheated. Parts AB and C were then added together with stirring. The resulting mixture was homogenised at 10000 rpm for 1 minute per 100 g, and then cooled to room temperature under stirring. Finally, Part D was added with gentle stirring.

The in-vitro SPF of the formulations was measured using the in vitro method of Diffey and Robson, J. Soc. Cosmet. Chem. Vol. 40, pp 127-133, 1989., using a spreadmaster robot. The formulations were prepared with and without the dispersion of the invention to determine the effect of the dispersion on the in-vitro SPF measurements.

TABLE 8
Formulations for Example 7

| | 7A | 7B | 7C | 7D | 7E | 7F |
|---|---|---|---|---|---|---|
| Ingredient | % w/w | % w/w | % w/w | % w/w | % w/w | % w/w |
| PART A | | | | | | |
| Arlacel 165 | 3.00 | 3.00 | 3.00 | 3.00 | 3.00 | 3.00 |
| Span 60 | 3.00 | 3.00 | 3.00 | 3.00 | 3.00 | 3.00 |
| Tween 60 | 0.40 | 0.40 | 0.40 | 0.40 | 0.40 | 0.40 |
| Crodacol S95 | 0.50 | 0.50 | 0.50 | 0.50 | 0.50 | 0.50 |
| Crodamol AB | 15.75 | 15.75 | 15.75 | 15.75 | 15.75 | 15.75 |
| Crodamol SSA | 8.00 | 8.00 | 8.00 | 8.00 | 8.00 | 8.00 |
| Xiameter | 2.00 | 2.00 | 2.00 | 2.00 | 2.00 | 2.00 |
| PMX-200 | | | | | | |
| Silicone | | | | | | |
| Fluid 350 cs | | | | | | |
| PART B | | | | | | |
| Solaveil CT-100 | 15.00 | 15.00 | — | — | — | — |
| Solaveil CZ-100 | 10.00 | 10.00 | — | — | — | — |
| Solaveil XT-100 | — | — | — | — | 20.00 | 20.00 |
| Tinosorb S | — | — | 2.00 | 2.00 | — | — |
| Eusolex OS | — | — | 5.00 | 5.00 | — | — |
| Eusolex 2292 | — | — | 8.00 | 8.00 | — | — |
| Uvinul A Plus | — | — | 5.00 | 5.00 | — | — |
| Dispersion of | — | 7.14 | — | 7.14 | — | 7.14 |
| Example 1 | | | | | | |
| Solaveil CT-12W | — | — | — | — | — | — |
| PART C | | | | | | |
| Water | 37.35 | 30.21 | 42.35 | 35.21 | 42.35 | 35.21 |
| Pricerine 9091 | 3.00 | 3.00 | 3.00 | 3.00 | 3.00 | 3.00 |
| Veegum Ultra | 0.80 | 0.80 | 0.80 | 0.80 | 0.80 | 0.80 |
| Keltrol KG-SFT | 0.20 | 0.20 | 0.20 | 0.20 | 0.20 | 0.20 |
| PART D | | | | | | |
| Euxyl K350 | 1.00 | 1.00 | 1.00 | 1.00 | 1.00 | 1.00 |

TABLE 9
in-vitro SPF measurements

| | | Standard | |
|---|---|---|---|
| Formulation | SPF | Dev | SPF Increase |
| 7A = Inorganic UV absorber only | 20 | 3.03 | — |
| 7B = 7A + Dispersion of Example 1 | 26 | 2.34 | 30% |
| 7C = Organic UV absorber only | 57 | 0.92 | — |
| 7D = 7C + Dispersion of Example 1 | 71 | 6.34 | 25% |
| 7E = Solaveil XT-100 | 8 | 1.17 | — |
| 7F = 7E + Dispersion of Example 1 | 11 | 0.33 | 38% |

The results show that the combinations of the dispersions according to the invention with UV absorbers or attenuators show an increase in the in-vitro SPF.

It is to be understood that the invention is not to be limited to the details of the above embodiments, which are described by way of example only. Many variations are possible.

Any or all of the disclosed features, and/or any or all of the steps of any method or process described, may be combined in any combination.

Each feature disclosed herein may be replaced by alternative features serving the same, equivalent or similar purpose. Therefore, each feature disclosed is one example only of a generic series of equivalent or similar features.

The above statements apply unless expressly stated otherwise. The term specification, for these purposes, includes the description and any accompanying claims, abstract and drawings.

1. A dispersion of precursor titanium dioxide particles having an intensity mean peak value particle size, measured by DLS as herein described, or a number mean peak value particle size, measured by DLS as herein described, in the range from 0.2 to 2.0 μm within a dispersing medium, wherein the particle size distribution of the titanium dioxide particles in dispersion is narrower than the particle size distribution of the precursor titanium dioxide particles.

2. A dispersion according to claim 1 wherein the DLS measurement is made using a Malvern Zetasizer.

3. A dispersion according to claim 1 wherein the dispersion contains at least 50% by weight of precursor titanium dioxide particles based on the total weight of the dispersion.

4. A dispersion according to claim 1 which is obtainable by mixing together the precursor titanium dioxide particles and the dispersing medium and milling the mixture at a rotational speed of between 2000 and 7000 r.p.m.

5. A dispersion according to claim 4 wherein the milling occurs in a horizontal bead mill.

6. A dispersion according to claim 1 wherein the dispersing medium is an organic medium.

7. A dispersion according to claim 1 wherein the dispersing medium comprises C12-C15 alkyl benzoate.

8. A dispersion according to claim 1 which further comprises a dispersing agent selected from polyglyceryl-3 polyricinoleate and polyhydroxystearic acid.

9. A dispersion according to claim 1 wherein the titanium dioxide particles in dispersion exhibit, measured by DLS and calculated as the difference between the number based d90 particle size and number based d10 particle size, at least a 2% narrower number based particle size distribution than the precursor titanium dioxide particles.

10. A dispersion according to claim 1 wherein the dispersion provides an increase in reflectance of infrared radiation, when measured as herein described by a N-IR spectrophotometer, compared to that of the precursor titanium dioxide particles.

11. A personal care composition comprising a dispersion according to claim 1.

12. A method of preparing a dispersion according to claim 1, the method comprising:
a) mixing the precursor titanium dioxide particles with a dispersing medium; and
b) milling the mixture to provide the dispersion.

13. A method for improving the infrared radiation reflecting properties of precursor titanium dioxide particles having an intensity mean peak value particle size, measured by DLS as herein described, or a number mean peak value particle size, measured by DLS as herein described, in the range from 0.2 to 2.0 μm, by preparing a dispersion of the precursor particles within a dispersing medium, wherein the distribution of particle sizes of the titanium dioxide particles in the dispersion is narrower than the distribution of particle sizes of the precursor titanium dioxide particles.

14. A method to attenuate infrared radiation in a cosmetic composition, the method comprising adding a dispersion according to claim 1 to the cosmetic composition.

15. A method to improve the SPF of a composition comprising a UV absorber, the method comprising adding a dispersion according to claim 1 to the composition.
